(19) United States
(12) Patent Application Publication
(10) Pub. No.: US 2026/0191865 A1
(43) Pub. Date: Jul. 9, 2026

(54) CANCER THERAPY WITH CAPIVASERTIB AND FULVESTRANT

(71) Applicant: AstraZeneca AB, Södertälje (SE)

(72) Inventors: Simon BARRY, Cambridge (GB); Andrew FOXLEY, Cambridge (GB); Amy MCDONOUGH, Cambridge (GB); Elza DE BRUIN, Cambridge (GB); Gaia SCHIAVON, Cambridge (GB)

(21) Appl. No.: 19/133,762

(22) PCT Filed: Dec. 6, 2023
(86) PCT No.: PCT/EP2023/084418

§ 371 Date: May 29, 2025

(30) Foreign Application Priority Data
Dec. 7, 2022 (GB) 2218395.8

(51) Int. Cl. A61K 31/519; A61K 31/565; A61P 35/00

(52) U.S. Cl. CPC A61K 31/519; A61K 31/565; A61P 35/00

(57) ABSTRACT

The present disclosure relates to therapeutic combinations of capivasertib and fulvestrant that are useful for treating specific populations of patients having advanced breast cancer, and to methods of treating specific populations of breast cancer patients with combinations of capivasertib and fulvestrant.

### FIELD OF THE INVENTION

The present disclosure relates to therapeutic combinations of capivasertib and fulvestrant that are useful for treating specific populations of patients having advanced breast cancer, and to methods of treating specific populations of breast cancer patients with combinations of capivasertib and fulvestrant.

### BACKGROUND

In women, breast cancer is both the most common cancer worldwide and the second cause of cancer death (Cardoso et al., Breast (2017) 31:244-259). Approximately 60% of premenopausal and 75% of postmenopausal breast cancer patients have estrogen receptor-positive (ER⁺) carcinomas. ER expression and activation are important factors to control tumour growth and recurrence (Chen, OMICS (2011)15:347-352). Although there are several therapeutic approaches, endocrine therapy has become the standard adjuvant treatment for postmenopausal women with ER⁺ breast cancer (Cardoso et al., Breast (2017) 31:244-259). Drugs that selectively target ER, like the selective ER down-regulators (SERDs), such as fulvestrant, or the selective ER modulators (SERMs), such as tamoxifen or drugs that prevent estrogen biosynthesis, like aromatase inhibitors (Als), are important therapeutic tools to block the ER signalling pathways that lead to cancer progression. Unless there is evidence of impending or actual visceral crisis, endocrine-based therapy is the preferred treatment modality because it has greater activity and better tolerability than cytotoxic chemotherapy. However, almost all tumours will become resistant to endocrine-based therapy and novel approaches are required to circumvent resistance, prolong the prechemotherapy window, and extend life.

The signalling pathway of phosphatidylinositol 3-kinase (PI3K)/protein kinase B (AKT) and mammalian target of rapamycin (mTOR) has been identified as playing a role in the development of resistance and is altered in approximately 50% of ER⁺ advanced breast cancer tumours. Mutations in exons 9 and 20 of PIK3CA, encoding the p110α subunit, are the most commonly found mutations but loss-of-function mutations in PTEN, a negative regulator of PI3K/AKT signalling, and activating mutations of AKT1 also occur. Increased activation of the PI3K/AKT pathway, via mTORC1 signalling, promotes tumour cell growth and survival, and results in ligand-independent activation of the ER and resistance to endocrine therapies (FIG. 1). Alternatively, inhibition of the PI3K pathway produces compensatory increases in ligand-dependent ER transcription and increased ER signalling.

Therefore, a rationale exists for simultaneously inhibiting the ER and PI3K/AKT pathway in patients with breast cancer.

AKT is a serine/threonine-specific protein kinase that plays a key role in multiple cellular processes such as glucose metabolism, apoptosis, cell proliferation, transcription, and cell migration. Mammalian cells express three closely related AKT isoforms that are encoded by different genes: AKT1 (protein kinase Bα), AKT2 (protein kinase Bβ), and AKT3 (protein kinase Bγ).

Capivasertib is a potent, selective pan-AKT kinase inhibitor that has shown activity in preclinical models of both endocrine-sensitive and endocrine-resistant BC when combined with the selective ER degrader (SERD) fulvestrant (Ribas R et al. Mol Cancer Ther (2015) 14:2035-48).

Fulvestrant, sold under the brand name FASLODEX among others, is used to treat ER+ metastatic breast cancer that may also be HER2-negative, and hormone receptor (HR)-positive, HER2-negative locally advanced or metastatic breast cancer in combination with palbociclib (a CDK4/6 inhibitor). It is a selective estrogen receptor degrader (SERD), which works both by down-regulating and by degrading the estrogen receptor.

The phase 2 FAKTION trial (NCT01992952) showed that the addition of capivasertib to fulvestrant endocrine therapy resulted in a significant improvement of progression-free survival (PFS) in postmenopausal women with aromatase inhibitor (AI)-resistant ER-positive, HER2-negative advanced breast cancer, and had no previous exposure to a cyclin-dependent kinase 4/6 (CDK4/6) inhibitor.

The FAKTION trial was originally designed in 2012, and defined PI3K/AKT/PTEN pathway-altered status in terms of whether tumours carried one of four specific PIK3CA mutations (E542K or E545K in exon 9 or H1047R or H1047L in exon 20 detected by either pyrosequencing or digital-droplet PCR [ddPCR] tests (or both) on tumour tissue or cell-free DNA [cfDNA]) or displayed loss of PTEN expression by immunohistochemistry. Using these original methods to identify tumour PI3K/AKT/PTEN pathway status, a secondary endpoint subgroup analysis suggested that the addition of capivasertib to fulvestrant conferred benefit for participants with either PI3K/AKT/PTEN pathway-altered advanced breast cancer or pathway non-altered advanced breast cancer (referred herein as the original pathway-altered and original pathway non-altered subgroups) (Jones R H et al., Lancet Oncol (2020) 21: 345-57).

A further evaluation of the FAKTION data analysed overall survival and an updated PFS analysis in the intent-to-treat population of FAKTION after an additional 34 months of follow-up. In a prespecified exploratory analysis, the investigators considered the benefit of capivasertib by tumour PI3K/AKT/PTEN pathway-altered status after expanding testing of the originally collected tumour or plasma samples to include next-generation sequencing (NGS) assays. The retrospective use of NGS testing identified an expanded pathway-altered subgroup of FAKTION participants whose tumours carried a PIK3CA mutation or AKT1 E17K or deleterious PTEN alteration, as well as the corresponding expanded pathway non-altered subgroup. The updated FAKTION data showed that the significant PFS and overall survival benefit of capivasertib was observed in the expanded PI3K/AKT/PTEN pathway-altered subgroup, but not in the expanded PI3K/AKT/PTEN pathway non-altered subgroup (Howell et al., Lancet Oncol (2022) 23: 851-64). The investigators hypothesised that the primary analysis (Jones R H et al., Lancet Oncol (2020) 21: 345-57) failed to detect the increased capivasertib benefit for participants with PI3K/AKT/PTEN pathway-altered tumours because limitations of the original tests erroneously placed some participants with bone fide pathway-altered tumours in the pathway non-altered subgroup.

No safety concerns were identified and FAKTION results led to the design and initiation of the phase 3 CAPItello-291 trial (NCT04305496). The aim of the phase 3 CAPItello-291 trial (NCT04035496) is to evaluate the efficacy and safety of capivasertib in combination with fulvestrant versus placebo with fulvestrant in patients with locally advanced or metastatic hormone receptor-positive/HER2-negative breast cancer, following recurrence or progression on or after AI therapy.

There is a need to further understand the impact of mutations in the PI3K/AKT/PTEN pathway on response to combined treatment with capivasertib and fulvestrant in the target patient populations.

### SUMMARY OF THE INVENTION

A first aspect of the invention provides a composition comprising capivasertib and a composition comprising fulvestrant for use as a combination therapy in the treatment of a patient having hormone receptor-positive (HR+) breast cancer, wherein the patient's tumour cells do not comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

A second aspect of the invention provides a method of treating a patient having hormone receptor-positive (HR+) breast cancer comprising administering to the patient a combination therapy comprising a therapeutically-effective amount of capivasertib and a therapeutically-effective amount of fulvestrant, wherein the patient's tumour cells do not comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

### DESCRIPTION OF THE DRAWINGS

FIG. 1 is a diagrammatic representation of the PI3K/AKT/PTEN and ER signalling pathways.

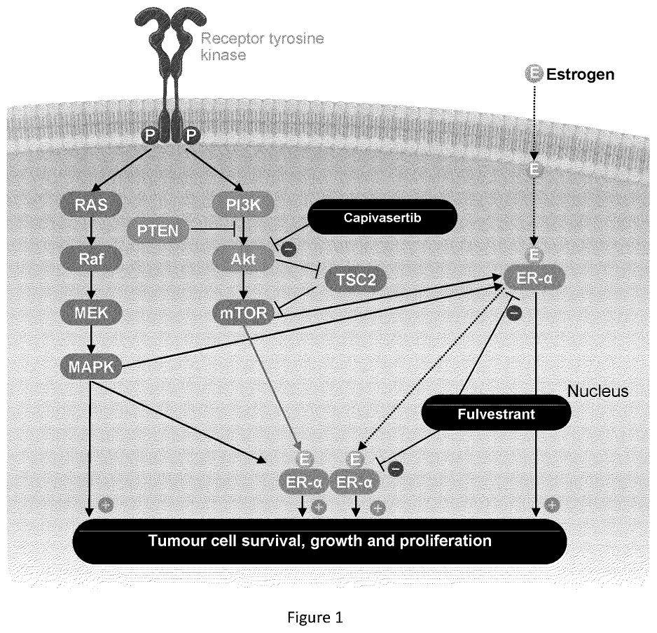

FIG. 2 is a graph showing progression-free survival (PFS) in the overall population. There were 258 PFS events in the Capivasertib+fulvestrant group (N=355), with a median PFS of 7.2 months (95% Cl: 5.5-7.4 months). There were 293 PFS events in the Placebo+fulvestrant group (N=353), with a median PFS of 3.6 months (95% Cl: 2.8-3.7 months). The adjusted hazard ratio (HR) was 0.60 (95% Cl: 0.51, 0.71; two-sided p-value<0.001). “+” in the figure indicates a censored observation. HR was estimated using the Cox proportional hazard model stratified by the presence of liver metastases, prior use of CDK4/6 inhibitor, and geographic region.

FIG. 3 is a graph showing PFS in the AKT pathway-altered population. There were 121 PFS events in the Capivasertib+fulvestrant group (N=155), with a median PFS of 7.3 months (95% Cl: 5.5-9.0 months). There were 115 PFS events in the Placebo+fulvestrant group (N=134), with a median PFS of 3.1 months (95% Cl: 2.0-3.7 months). The adjusted hazard ratio (HR) was 0.50 (95% Cl: 0.38, 0.65; two-sided p-value<0.001). “+” in the figure indicates a censored observation. HR was estimated using the Cox proportional hazard model stratified by the presence of liver metastases and prior use of CDK4/6 inhibitor.

FIG. 4 is a graph showing PFS in the pathway non-altered population (including unknowns, i.e. patients with no valid NGS results). There were 137 PFS events in the Capivasertib+fulvestrant group (N=200), with a median PFS of 7.2 months (95% Cl: 4.5-7.4 months). There were 178 PFS events in the Placebo+fulvestrant group (N=219), with a median PFS of 3.7 months (95% Cl: 3.0-5.0 months). The hazard ratio (HR) was 0.70 (95% Cl: 0.56, 0.88). “+” in the figure indicates a censored observation.

FIG. 5 is a graph showing PFS in the pathway non-altered population, (excluding unknowns). There were 103 PFS events in the Capivasertib+fulvestrant group (N=142), with a median PFS of 5.3 months (95% Cl: 3.6-7.3 months). There were 141 PFS events in the Placebo+fulvestrant group (N=171), with a median PFS of 3.7 months (95% Cl: 3.5-5.1 months). The hazard ratio (HR) was 0.79 (95% Cl: 0.61, 1.02). “+” in the figure indicates a censored observation.

### DETAILED DESCRIPTION

The present disclosure relates to the surprising finding that a combination therapy comprising capivasertib and fulvestrant is useful for treating hormone receptor-positive (HR+) advanced breast cancer in a population of patients whose tumour tissue does not comprise any of a number of specific genetic mutations affecting the PI3K/AKT/PTEN pathway.

In particular, the present inventors have shown that a combination therapy comprising capivasertib and fulvestrant can be used to increase progression-free survival (PFS) in a patient population that do not have specific genetic mutations affecting the PI3K/AKT/PTEN pathway.

A first aspect of the present invention provides a pharmaceutical composition comprising capivasertib and a pharmaceutical composition comprising fulvestrant for use as a combination therapy in the treatment of a patient having hormone receptor-positive (HR+) breast cancer, wherein the patient's tumour cells do not comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

A second aspect of the invention provides a method of treating a patient having an hormone receptor-positive (HR+) breast cancer comprising administering to the patient a combination therapy comprising a therapeutically-effective amount of capivasertib and a therapeutically-effective amount of fulvestrant, wherein the patient's tumour cells do not comprise any of the following mutations:
i. E17K in the AKT1 gene;
ii. Any of the mutations in the PIK3CA gene listed in Table 2; and iii. Any of the mutations in the PTEN gene listed in Table 3 or in Table 4.

The human wild-type P/K3CA, AKT1 and PTEN genes are identified in Table 1.

TABLE 1

| | | Ensemble | |
|---|---|---|---|
| Gene | Full Name | Transcript | Refseq |
| PIK3CA | phosphatidylino- | ENST00000263967 | NM_006218.2 |
| | sitol-4,5- | | |
| | bisphosphate | | |
| | 3-kinase, catalytic | | |
| | subunit alpha | | |
| AKT1 | AKT serine/ | ENST00000555528.5 | NM_005163 |
| | threonine kinase 1 | | |
| PTEN | phosphatase and | ENST00000371953 | NM_000314.4 |
| | tensin homolog | | |

As P/K3CA and AKT1 are oncogenes, mutations that result in activation of the protein affect the PIK3CA/AKT1/PTEN pathway. A list of qualifying mutations in AKT1 and P/K3CA genes is provided in Table 2.

TABLE 2

| Gene | Mutation | Exon | Comments |
|---|---|---|---|
| AKT1 | E17K | 2 | Hotspot mutation |
| PIK3CA | R88Q | 1 | PI3K-ABD (p85) binding domain |
| PIK3CA | N345K | 4 | |
| PIK3CA | C402R | 7 | |
| PIK3CA | E542K | 9 | Helical domain |
| PIK3CA | E545A | 9 | Helical domain |
| PIK3CA | E545D | 9 | Helical domain |
| PIK3CA | E545Q | 9 | Helical domain |
| PIK3CA | E545K | 9 | Helical domain |
| PIK3CA | E545G | 9 | Helical domain |
| PIK3CA | Q546E | 9 | Helical domain |
| PIK3CA | Q546K | 9 | Helical domain |
| PIK3CA | Q546R | 9 | Helical domain |
| PIK3CA | Q546P | 9 | Helical domain |
| PIK3CA | M1043V | 20 | PI3_PI4_Kinase domain |
| PIK3CA | M1043I | 20 | PI3_PI4_Kinase domain |
| PIK3CA | H1047Y | 20 | PI3_PI4_Kinase domain |
| PIK3CA | H1047R | 20 | PI3_PI4_Kinase domain |
| PIK3CA | H1047L | 20 | PI3_PI4_Kinase domain |
| PIK3CA | G1049R | 20 | PI3_PI4_Kinase domain |

PTEN is a tumour suppressor gene, therefore gene alterations that result in loss of a functional protein affect the PIK3CA/AKT1/PTEN pathway. The present inventors have generated a list of seven distinct criteria to identify such alterations by next-generation sequencing (NGS). Details of the criteria to identify qualifying alterations in the PTEN gene are provided in Table 3, and further specific qualifying missense mutations are provided in Table 4.

TABLE 3

| Inclusion criteria | |
|---|---|
| for deleterious | |
| PTEN alterations | Additional information |
| Nonsense mutation | Any nonsense mutation leads to interrupted |
| i.e. creates a STOP codon | translation/nonsense-mediated decay (NMD). |
| Frameshift Mutation | Any frameshift mutation usually leads to |
| i.e. insertion or deletion | interrupted translation/NMD. |
| causing a frameshift | |
| in the coding sequence | |
| Splicing Mutation | Mutations or deletion of any of the 4 bases |
| | that immediately flank coding exons |
| | (i.e. −2, −1, +1 or +2 to a coding |
| | exon) are expected to lead to incorrect |
| | splicing, which likely leads to a non- |
| | functional protein. |
| Start Codon Mutation | Any change to the recognised start codon |
| | (ATG/Methionine 1) for the main transcript |
| | should lead to loss of protein translation. |
| Deep deletion | Partial or entire PTEN homozygous gene |
| | deletion qualifies. |
| Intragenic Rearrangement | Any large-scale change within the PTEN |
| i.e. any rearrangement of | gene considered to be disruptive, including |
| the PTEN locus clearly | rearrangements and fusion (with another |
| disruptive to the gene. | gene) Note: duplication, gain or |
| | amplification do not qualify. |

TABLE 4

| | Protein | | |
|---|---|---|---|
| Gene | change | exon | Additional information |
| PTEN | C124R | 5 | Associated with Cowden Syndrome; |
| | | | in phosphatase p-loop |
| PTEN | C124S | 5 | In phosphatase p-loop |
| PTEN | G129E | 5 | In phosphatase p-loop |
| PTEN | G129V | 5 | In phosphatase p-loop |
| PTEN | G129R | 5 | In phosphatase p-loop |
| PTEN | R130Q | 5 | Associated with Cowden Syndrome, |
| | | | commonly mutation position across |
| | | | multiple tumour types |
| PTEN | R130G | 5 | Associated with Cowden Syndrome, |
| | | | commonly mutation position across |
| | | | multiple tumour types |
| PTEN | R130L | 5 | Associated with Cowden Syndrome, |
| | | | commonly mutation position across |
| | | | multiple tumour types |
| PTEN | R130P | 5 | Associated with Cowden Syndrome, |
| | | | commonly mutation position across |
| | | | multiple tumour types |
| PTEN | C136R | 5 | Associated with hereditary cancer- |
| | | | predisposing syndrome, associated |
| | | | with hamartoma tumour syndrome |
| PTEN | C136Y | 5 | Associated with Cowden Syndrome, |
| | | | associated with hereditary cancer- |
| | | | predisposing syndrome |
| PTEN | S170R | 6 | in TI-loop |
| PTEN | R173C | 6 | Located at the phosphatase-C2 domain |

The sample obtained from the patient may be any sample type that contains breast tumour genomic material (e.g. tissue, blood, plasma or cell-free DNA). Preferably, the sample is a breast tumour tissue sample.

There are a variety of methods which are routinely used in the art to detect genetic mutations, and any suitable method can be used.

Next-generation sequencing (NGS) technologies can detect hundreds of alterations across multiple genes in a single test, and as the skilled person will be aware, NGS can be used to define tumour biomarker status. A single NGS assay can sensitively detect activating PIK3CA mutations and AKT1 mutations across their entire gene structures, as well as PTEN alterations and gene deletion.

In a preferred embodiment, NGS is used to detect the presence or absence of any of the mutations detailed in Tables 2-4 in a sample containing tumour cells obtained from the patient. Preferably, the sample is a breast tumour tissue sample.

Commercially available NGS technologies include the FoundationOne®CDx (F1CDx) NGS Clinical Trial Assay from Foundation Medicine, Cambridge, MA, USA), which can be used to detect single-nucleotide variations, insertion and deletion alterations, and copy number alterations in DNA isolated from formalin-fixed paraffin-embedded tumour tissue specimens. The GuardantOMNI™ (Guardant Health, Redwood City, CA, USA) detects single-nucleotide variations, insertion and deletion alterations, copy number alterations, or fusions in 500 genes, including PIK3CA, AKT1, and PTEN alterations, using NGS of cfDNA extracted from plasma samples. Burning Rock Biotech Limited (Guangzhou, China) is developing a liquid biopsy approach, with NGS-based circulating tumour DNA (ctDNA) assays.

Jones R H et al (Lancet Oncol (2020) 21: 345-57) describe using pyrosequencing and/or digital-droplet PCR [ddPCR] tests on tumour tissue or cell-free DNA [cfDNA]) or displayed loss of PTEN expression by immunohistochemistry to identify tumour PI3K/AKT/PTEN pathway status.

As used herein, the terms “patient” and “subject” are used interchangeably and refer to a mammal, and preferably to a human. The patient may be a pre- or post-menopausal woman, or a man.

The patient has a hormone receptor-positive (HR+) breast cancer, meaning that the tumour cells express surface receptors that bind to the hormones estrogen and/or progesterone.

In one embodiment, the patient has estrogen receptor-positive (ER+) breast cancer (with or without co-expression of progesterone receptor). ER+ cancer can be defined as at least 10% of primary tumour or metastatic tumour cells staining positive for the estrogen receptor.

In a preferred embodiment the cancer is classified as a HER2-negative cancer, meaning that the tumour cells do not express human epidermal growth factor receptor 2 (HER2). This is histologically confirmed from biopsy taken at diagnosis or from metastasis. In one embodiment, HER2-negative is defined as immunohistochemistry (IHC) scores of 0, 1+, or 2+ and in situ hybridisation (ISH)-negative.

In a preferred embodiment the cancer is classified as an advanced breast cancer (ABC), meaning histologically confirmed, locally advanced (inoperable) or metastatic breast cancer, with radiological or objective evidence of recurrence or progression. either with recurrence or progression while on, or within 12 months of, the end of (neo)adjuvant treatment with a regimen containing an aromatase inhibitor (AI) either as a single agent or in combination.

Aromatase inhibitors (Als) are one of the principal therapeutic approaches for estrogen receptor-positive (ER⁺) breast cancer in postmenopausal women. They block estrogen biosynthesis through aromatase inhibition, thus preventing tumour progression. Examples of Als include anastrozole (sold under the brand name ARIMIDEX, among others), exemestane (sold under the brand name AROMASIN, among others) and letrozole (sold under the brand name FEMARA, among others).

The patient may or may not have been previously treated with a CDK4/6 inhibitor (e.g. (Palbociclib (sold under the brand name IBRANCE among others), ribociclib (sold under the brand names KISQALI and KRYXANA), abemaciclib (sold under the brand name VERZENIO among others)). CDK4/6 inhibitors are, in certain markets, approved treatment options in combination with an aromatase inhibitor or fulvestrant for patients with advanced or metastatic HR+HER2− breast cancer who have received prior endocrine therapy and/or as initial endocrine-based therapy. In one embodiment, the patient has been previously treated with a CDK4/6 inhibitor.

According to the invention, therapeutically effective amounts of capivasertib and fulvestrant can be used to treat advanced breast cancer in a patient.

Capivasertib (also known as AZD5363 and by the chemical name of (S)-4-amino-N-(1-(4-chlorophenyl)-3-hydroxypropyl)-1-(7H-pyrrolo[2,3-d]pyrimidin-4-yl)piperidine-4-carboxamide) is an investigational oral treatment currently in Phase Ill trials for the treatment of multiple subtypes of breast cancer, prostate cancer and a Phase II trial for haematologic malignancies. A potent, selective adenosine triphosphate (ATP)-competitive inhibitor of all three AKT isoforms (AKT1/2/3), capivasertib is being evaluated in combination with existing therapies in tumours harbouring alterations in the PI3K/AKT/PTEN pathway, and in tumours reliant on signalling via this pathway for survival.

In a preferred embodiment, capivasertib is administered according to the following dosage regimen: 400 mg orally twice daily; 4 days on, 3 days off.

Fulvestrant, sold under the brand name FASLODEX among others, and known by the chemical name 7α-[9-[(4,4,5,5,5-Pentafluoropentyl)-sulfinyl]nonyl]estra-1,3,5(10)-triene-3,17β-diol, is a selective estrogen receptor degrader (SERD) used to treat ER+ metastatic breast cancer (including ER+/HER2-negative breast cancers). Fulvestrant is preferably administered by intramuscular injection and is provided in a pre-filled syringe containing 250 mg fulvestrant in 5 ml solution. The recommended dose in female adults is 500 mg at intervals of one month, with an additional 500 mg dose given two weeks after the initial dose.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “treat,” “treating,” and “treatment” refer to at least partially alleviating, inhibiting, preventing and/or ameliorating a condition, disorder, or disease, such as advanced breast cancer. The effectiveness of treatment of advanced breast cancer can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging progression free survival (PFS).

The term “combination therapy” can refer to simultaneous, separate, or sequential administration of two or more therapeutic agents. In one embodiment, “combination” can refer to simultaneous administration (e.g., administration of both agents in a single dosage form). In another embodiment, “combination” refers to separate administration (e.g., administration of both agents in separate dosage forms, but at substantially the same time). In a further, and preferred, embodiment of the invention, “combination” refers to separate and sequential administration (e.g., where a first therapeutic agent is administered, followed by a delay, followed by administration of a second or further therapeutic agent). The two therapeutic agents (Capivasertib and fulvestrant) may each be administered multiple times within a pre-defined treatment cycle. Where the administration is sequential or separate, the delay in administering the later component should be neither too long nor too short, so as not to lose the benefit of the combination.

The terms “co-administration,” “in combination with,” “simultaneous,” and “concurrent,” as used herein, encompass administration of two or more active pharmaceutical ingredients to a subject and include simultaneous administration in separate compositions, administration at different times in separate compositions, or administration in a composition in which two or more active pharmaceutical ingredients are present.

The present inventors have shown that a combination therapy comprising capivasertib and fulvestrant can be used to increase progression-free survival (PFS) in a patient population that do not have specific genetic mutations affecting the PI3K/AKT/PTEN pathway. In an embodiment, the median PFS in the patient population treated with the combination therapy is 4 months or greater, 5 months or greater, or about 5.3 months.

As used herein in the context of a clinical trial study, the term “progression free survival (PFS)” is defined as the time (usually measured in months) from randomisation to either the first documented progression confirmed by RECIST version 1.1 criteria (see Eisenhauer et al., European Journal of Cancer (2009) 45:228-247) or death from any cause. In a real-world clinical (non-trial) setting, PFS can be defined as the time from first administration of the combination therapy to either the first documented progression confirmed by RECIST version 1.1 criteria or death from any cause.

By administering a combination of capivasertib and fulvestrant to a patient in accordance with the present invention as defined herein, PFS can be increased by at least 20%, 30%, 40%, 50%, 60%, 70%, 80%, 90%, 100%, 110%, 120%, 130% 140%, 150% compared to treatment with fulvestrant without capivasertib. In an embodiment, the increase in PFS time is clinically meaningful. In another embodiment, the increase in PFS time is statistically significant.

A further benefit of the use of the combination therapy comprising capivasertib and fulvestrant in a patient population that do not have specific genetic mutations affecting the PI3K/AKT/PTEN pathway is an increase in overall survival (OS), defined as the time (usually measured in months) from randomisation to death from any cause. In a real-world clinical (non-trial) setting, OS can be defined as the time from administration of the combination therapy to death from any cause.

By administering a combination of capivasertib and fulvestrant to a patient in accordance with the present invention as defined herein, OS can be increased by at least 20%, 30%, 40%, 50%, 60%, 70%, 80%, 90%, 100%, 110%, 120%, 130% 140%, 150% compared to treatment with fulvestrant without capivasertib.

### MODES FOR CARRYING OUT THE INVENTION

The invention will now be further defined by reference to the following non-limiting example.

### Example

### Methods

Eligible pre- or post-menopausal women or men with HR+/HER2− advanced breast cancer that had recurred or progressed on or within 12 months after AI therapy with or without CDK4/6 inhibitor were randomized 1:1 to receive fulvestrant (F) (per standard dosing schedule (intramuscular injection of 500 mg dose: 28-day cycle 1, days 1 & 15; then every 4 weeks)) with either placebo (PBO) or capivasertib (400 mg orally twice daily; 4 days on, 3 days off).

Randomization was stratified by the presence of liver metastases, prior use of CDK4/6 inhibitors and geographic location.

Treatment was continued until objective radiological disease progression as defined by Response Evaluation Criteria in Solid Tumors version 1.1, unacceptable toxicity, withdrawal of consent, or death.

Given the importance of AKT pathway activation, patients were assigned as AKT pathway-altered post-randomization based on next-generation sequencing analysis identifying at least one qualifying P/K3CA, AKT1, or PTEN alteration in tumor tissue collected before randomization.

Qualifying P/K3CA and AKT1 alterations are detailed in Table 2 above. Qualifying PTEN alterations are detailed in Tables 3 and 4 above. If tumour cells in the sample obtained from the patient are identified as having any one or more of the genetic mutations listed in any of Tables 2-4 then the patient is characterised as having a PI3K/AKT/PTEN pathway-altered status. If tumour cells are identified as not having any of the genetic mutations listed in any of Tables 2-4 then the patient is characterised as having a PI3K/AKT/PTEN pathway non-altered status.

The dual primary endpoint was investigator-assessed progression-free survival (PFS) in the overall population (i.e. pathway-altered and non-pathway altered subjects, including unknowns) and in the pathway-altered population.

### Results

A total of 708 patients were randomized: 355 to capivasertib+F and 353 to PBO+F. Overall, 41% of patients had AKT pathway alterations, determined centrally using next-generation sequencing in tumour tissue with the FoundationOne®CDx assay (and using Burning Rock assay for the test cohort in China). 22% of patients were pre/peri menopausal and 77% post-menopausal women, 1% male. 87% of patients had ≥1 line of prior disease-related therapy for locally advanced or metastatic disease: 69% had received CDK4/6 inhibitor and 18% had received prior chemotherapy. Demographic and baseline characteristics were broadly balanced between the overall and altered populations, and by treatment arms.

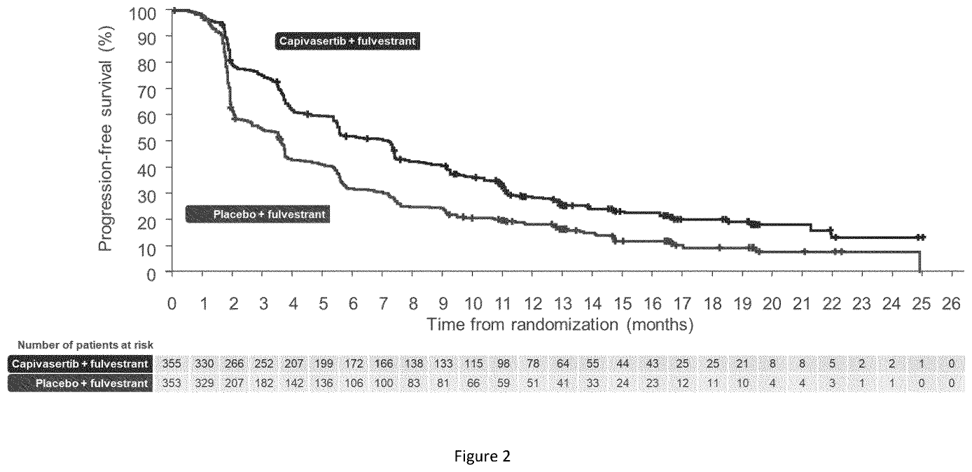

At primary analysis, 551 PFS events had occurred in the overall population (see FIG. 2). PFS was significantly longer with capivasertib+F vs PBO+F (hazard ratio [HR]0.60; 95% confidence interval [Cl]0.51-0.71; p<0.001; median 7.2 vs 3.6 months).

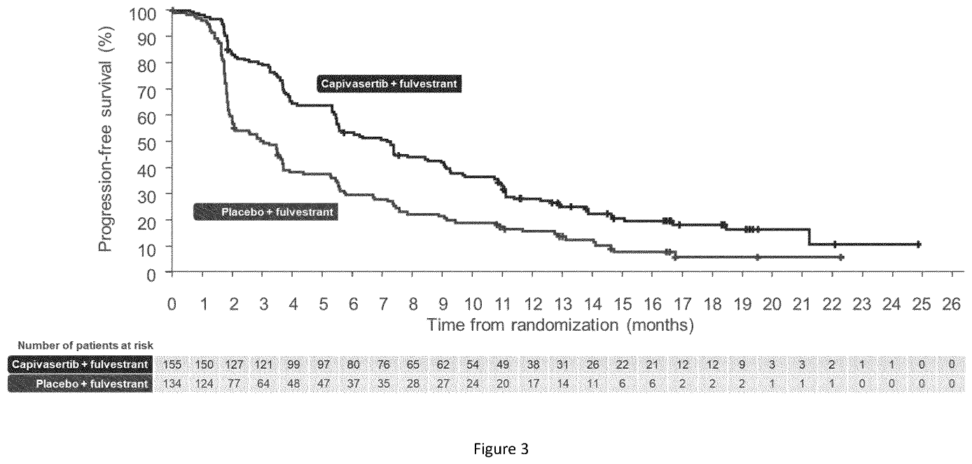

236 PFS events had occurred in the AKT pathway-altered population (see FIG. 3). PFS was significantly longer with capivasertib+F vs PBO+F (HR 0.50; 95% Cl 0.38-0.65; p<0.001; median 7.3 vs 3.1 months).

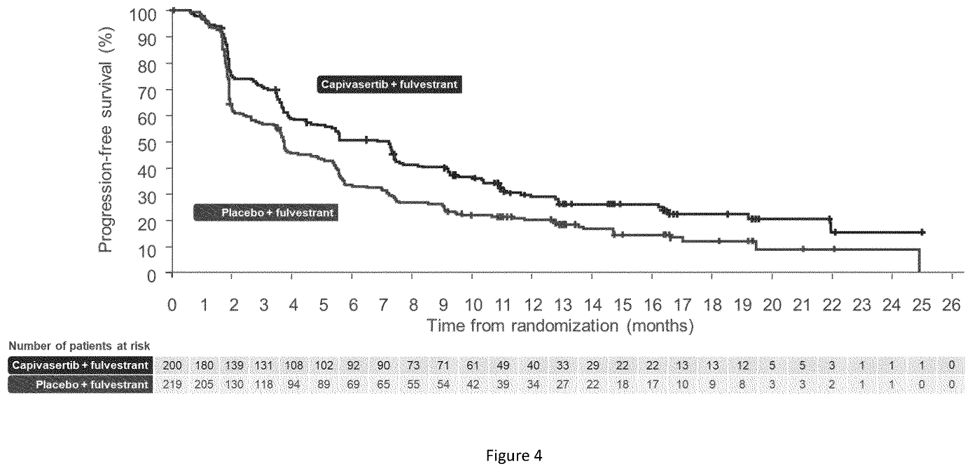

For the pathway non-altered population (including unknowns (i.e. patients with no valid NGS results)) 315 PFS events had occurred (see FIG. 4). Again, PFS was found to be significantly longer with capivasertib+F vs PBO+F in the AKT pathway non-altered population (HR 0.7; 95% Cl 0.56-0.88; median 7.2 vs 3.7 months).

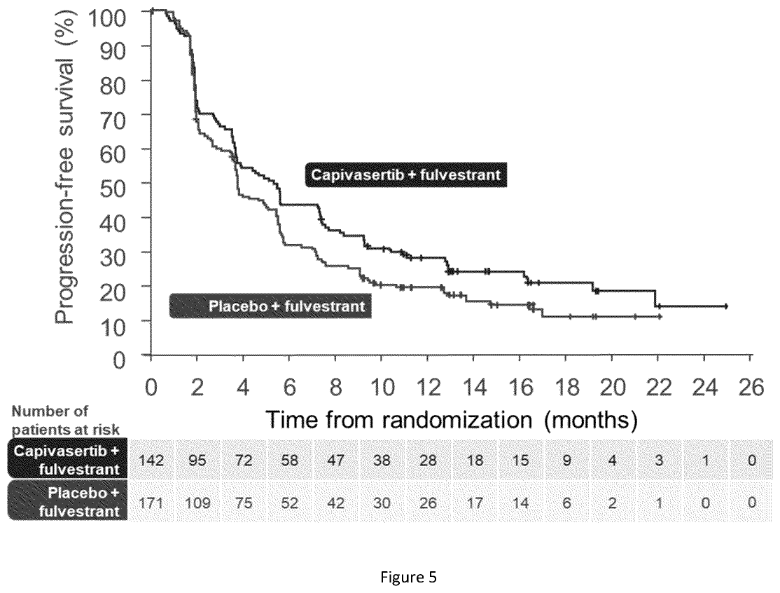

For the pathway non-altered population (excluding unknowns) 144 PFS events occurred (see FIG. 5). Again, PFS was found to be significantly longer with capivasertib+F vs PBO+F (HR 0.79; 95% Cl 0.61-1.02; median 5.3 vs 3.7 months).

The longer PFS with capivasertib+F vs PBO+F was found to be similar for those patients in the overall population with prior use of CDK4/6 inhibitors (HR 0.62; 95% Cl 0.51-0.75) and those patients in the overall population without prior use of CDK4/6 inhibitors (HR 0.65; 95% Cl 0.47-0.91).

Objective response rate among patients with measurable disease in the capivasertib+F vs PBO+F arms was 22.9% vs 12.2% in the overall population and 28.8% vs 9.7% in the altered population. In the overall population, the most frequent all-grade adverse events (AEs; unadjusted for exposure) with capivasertib+F were diarrhea (72.4% vs 20.0% PBO+F arm), nausea (34.6% vs 15.4%) and rash (22.0% vs 4.3%). The most frequent Grade ≥3 AEs were diarrhea (9.3% vs 0.3%), rash maculo-papular (6.2% vs 0%) and rash (5.4% vs 0.3%). AEs leading to discontinuation of capivasertib/placebo were reported in 13.0% and 2.3% of patients, respectively.

### CONCLUSIONS

This phase 3 clinical trial met both primary endpoints with capivasertib+F significantly improving PFS in both the AKT pathway-altered and in the AKT pathway non-altered populations.

This is a particularly significant outcome having therapeutic implications for patients in the pathway non-altered population, since an earlier study found that in the NGS-identified pathway non-altered subgroup PFS was similar for the both the capivasertib+F and PBO+F treatment groups. While the significant PFS and overall survival benefit of capivasertib was seen in the pathway-altered subgroup, no such benefit was seen in the pathway non-altered subgroup (see Howell et al., Lancet Oncol 2022; 23: 851-64).

The overall safety profile of capivasertib+F appears consistent with the known profile of the combination. This is the first study to show statistically significant, clinically meaningful PFS improvement with an AKT inhibitor in HR+ABC.

1. A composition comprising capivasertib and a composition comprising fulvestrant for use as a combination therapy in the treatment of a patient having hormone receptor-positive (HR+) breast cancer, wherein the patient's tumour cells do not comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

2. A composition comprising capivasertib and a composition comprising fulvestrant for use according to claim 1, wherein the patient has estrogen receptor-positive (ER+) breast cancer.

3. A composition comprising capivasertib and a composition comprising fulvestrant for use according to claim 1 or claim 2, wherein the patient has an aromatase inhibitor-resistant breast cancer.

4. A composition comprising capivasertib and a composition comprising fulvestrant for use according to any preceding claim, wherein the patient has a HER2-negative breast cancer.

5. A composition comprising capivasertib and a composition comprising fulvestrant for use according to any preceding claim, wherein the composition comprising capivasertib and the composition comprising fulvestrant are administered separately.

6. A method of treating a patient having hormone receptor-positive (HR+) breast cancer comprising administering to the patient a combination therapy comprising a therapeutically-effective amount of capivasertib and a therapeutically-effective amount of fulvestrant, wherein the patient's tumour cells do not comprise any of the following mutations:
i. E17K in the AKT1 gene;
ii. Any of the mutations in the PIK3CA gene listed in Table 2; and
iii. Any of the mutations in the PTEN gene listed in Table 3 or in Table 4.

7. A method according to claim 6, wherein the patient has estrogen receptor-positive (ER+) breast cancer.

8. A method according to claim 6 or claim 7, wherein the patient has an aromatase inhibitor-resistant breast cancer.

9. A method according to any of claims 6 to 8, wherein the patient has a HER2-negative breast cancer.

10. A method according to any of claims 6 to 9, wherein capivasertib and fulvestrant are administered separately.
